(19) United States
(12) Patent Application Publication
(10) Pub. No.: US 2024/0185982 A1
(43) Pub. Date: Jun. 6, 2024

(54) MEDICAL CARE ASSISTANCE SYSTEM, MEDICAL CARE ASSISTANCE DEVICE, AND RECORDING MEDIUM

(71) Applicant: OMRON HEALTHCARE Co., Ltd., Kyoto (JP)

(72) Inventors: Fumihiko NAKAMURA, Kyoto (JP); Ai KANAZAWA, Kyoto (JP); Hiroshi USUI, Kyoto (JP)

(21) Appl. No.: 18/442,707

(22) Filed: Feb. 15, 2024

Related U.S. Application Data
(63) Continuation of application No. PCT/JP2023/007623, filed on Mar. 1, 2023.

(30) Foreign Application Priority Data
Mar. 31, 2022 (JP) 2022-060885

(51) Int. Cl. G16H 20/30; G16H 30/20

(52) U.S. Cl. CPC G16H 20/30; G16H 30/20

(57) ABSTRACT

A medical care assistance system including a minimum exercise intensity calculation means configured to obtain a minimum exercise intensity in a first predetermined period, an estimated severity information calculation means configured to obtain, based on the minimum exercise intensity, estimated severity information, a medical care assistance image generation means configured to generate a medical care assistance image including an estimated severity time series graph indicating, as a time axis, a second predetermined period, and indicating, along the time axis, an estimated severity display bar such that the estimated severity information of a period is identifiably characteristically displayed for each of the first predetermined period or an alternative first predetermined period that is a period alternative to the first predetermined period, and an output means configured to output the medical care assistance image.

### CROSS-REFERENCE TO RELATED APPLICATIONS

This application is the U.S. national stage application filed pursuant to 35 U.S.C. 365(c) and 120 as a continuation of International Patent Application No. PCT/JP2023/007623, filed Mar. 1, 2023, which application claims priority to Japanese Patent Application No. 2022-060885, filed Mar. 31, 2022, which applications are incorporated herein by reference in their entireties.

### TECHNICAL FIELD

The present invention belongs to a technical field related to healthcare, and relates to a medical care assistance system, a medical care assistance device, and a program.

### BACKGROUND ART

In recent years, there has been proposed a system that continuously acquires and records biological information of a patient and indicates transition overtime in the biological information to assist medical care by a physician (for example, Patent Document 1).

In Patent Document 1, a medical information processing system is disclosed which stores vital data of a patient in association with time, displays the vital data in time series, and calculates and displays statistical information related to the vital data displayed in time series. According to this system, an operator such as a physician can easily comprehend the tendency of the vital data of the patient, comprehend the condition of the patient, and determine the type and dosage of medication to be prescribed to the patient.

By using such a system, it is possible to reduce a burden on a physician in the medical care of a patient, in particular the patient having a chronic disease, and if an appropriate treatment strategy can be quickly determined, the effect will be exerted on the patient.

### CITATION LIST

### Patent Literature

Patent Document 1: JP 2019-24943 A

### SUMMARY OF INVENTION

### Technical Problem

In diagnosis and treatment of heart failure, which a major chronic disease, it is common practice to evaluate severity (degree of progression) of the disease based on subjective symptoms produced by various daily physical activities, using the New York Heart Association functional classification (NYHA classification) or the like. Then, the physician determines the type and dosage of medication to be prescribed and other treatment strategies in accordance with the evaluated severity.

In the related art, a physician has classified a patient into such a classification described above by the physician performing medical interview and determination at the medical examination on the patient about what kind of physical activity caused (or not caused) a significant (painful) symptom to appear. In such a method, there is a problem in that it is difficult to accurately obtain information from the patient and perform accurate classification (evaluation) in a limited medical examination time. In addition, from the standpoint of the patient, it is stressful for the patient to be forced the same questions and answers every time the outpatient medical examination is performed, and the burden on the patient is large.

In the known medical care assistance system as disclosed in Patent Document 1, although transition information of the vital data such as a pulse rate and a blood pressure value of the patient can be indicated, there is a problem that it is not possible to assist the classification of the severity based on the physical activity and the subjective symptom as described above.

In view of the problem as described above, the present invention relates to a system related to medical assistance, and an object of the present invention is to provide a technique capable of reducing a workload on a medical professional to perform diagnosis including severity of a disease of medical care target for a patient.

### Solution to Problem

The present invention adopts the following configurations to solve the above-described problems. That is, a medical care assistance system including a minimum exercise intensity calculation means configured to obtain a minimum exercise intensity of an exercise having the smallest exercise intensity among exercises that make a symptom related to a disease of medical care target for a patient to appear within a first predetermined period, an estimated severity information calculation means configured to obtain, based on the minimum exercise intensity, estimated severity information indicating estimated severity of a disease of the patient, a medical care assistance image generation means configured to generate a medical care assistance image including an estimated severity time series graph indicating, as a time axis, a second predetermined period that is a past predetermined period including the first predetermined period, and indicating, along the time axis, an estimated severity display bar such that the estimated severity information is identifiably characteristically displayed for each of the first predetermined period or an alternative first predetermined period that is a period alternative to the first predetermined period, and an output means configured to output the medical care assistance image.

Here, the “first predetermined period” may be any period as long as it is a period in which the patient can review the physical activity without a burden and/or a period in which it is easy to comprehend a change in the severity or transition in the symptom, and can be set to, for example, one week (7 days). In addition, the “second predetermined period” is, for example, a period in which the transition of the disease condition of the patient can be checked at a glance, and may be, for example, one month (or four weeks). That is, the second predetermined period may include a plurality of the first predetermined periods. Further, the first predetermined period included in the second predetermined period in the estimated severity time series graph may not necessarily include the latest first predetermined period. In addition, the severity (of the disease) mentioned here includes a severity classification or the like defined by a medical care guideline or the like of the disease, and also includes a degree of deterioration of a body function. The “exercise intensity” can be indicated by using an index such as METs. The METs is an indicator of an activity intensity indicating how many times more energy is consumed by various activities with respect to a resting state (a state of sitting quietly) as a 1 METs. The “output means” may be a display device such as a liquid crystal display, or a printing device such as a printer.

According to such a configuration, the physician can refer to the transition of the estimated severity of the disease of medical care target for the patient by referring to the medical care assistance image to be output. Accordingly, for example, by acquiring (inputting) necessary information prior to the medical examination of the patient and referring in advance to the medical care assistance image generated based on the acquired information, futile questions can be eliminated from the contents of questions for checking the transition of the disease condition of the patient and questions for diagnosing the severity at the time of the medical examination, and appropriate medical examination can be performed more efficiently. In addition, since it is possible to suppress a regular and redundant medical interview for every medical examination, it is possible to contribute to a reduction in the burden on the patient.

The medical care assistance system may further include a symptom appearing exercise information acquisition means configured to acquire symptom appearing exercise information which is information including a content of exercise that caused a symptom related to the disease to appear, and the minimum exercise intensity calculation means may calculate the minimum exercise intensity based on the symptom appearing exercise information. According to such a configuration, it is possible to easily obtain the minimum exercise intensity based on the information of the daily occurrence of the symptom of the patient and the exercise content at that time.

The symptom appearing exercise information acquisition means may further acquire information regarding the presence or absence of the symptom related to the disease of the patient for each third predetermined period, and the medical care assistance image generation means may generate the medical care assistance image displaying, along the time axis, information regarding the presence or absence of the symptom for each of the third predetermined periods.

Here, the “third predetermined period” is desirably a period suitable for reviewing the presence or absence of a symptom in accordance with a certain constant life activity cycle, and can be set to one day (24 hours), for example. According to such a configuration, by referring to the medical care assistance image, the physician can check the transition in feeling of subjective symptom by the patient for each day (the kind and frequency thereof) together with the transition of the estimated severity, and can more appropriately diagnose the disease condition of the patient.

In the estimated severity time series graph, a length of a period indicated by one of the estimated severity display bars may have the third predetermined period as a minimum value.

The symptom appearing exercise information acquisition means may include, for example, an input means (for example, a keyboard, a mouse, and a touch panel) of an information processing terminal used by a medical professional such as a physician. That is, the medical professional may input the symptom appearing exercise information by asking the patient at a medical institution, a patient's home, or the like. Further, the symptom appearing exercise information acquisition means may include a means that requests the patient (or a caregiver thereof) to input the symptom appearing exercise information.

That is, the medical care assistance system may further include an automatic medical interview terminal configured to execute an automatic medical interview processing of requesting the patient to input patient information including at least the symptom appearing exercise information of the patient within the most recent first predetermined period, and the symptom appearing exercise information acquisition means may acquire the symptom appearing exercise information input from the patient through an automatic medical interview processing executed in the automatic medical interview terminal. The automatic medical interview processing may include a notice to prompt the patient to input the patient information according to a predetermined schedule (for example, each time the first predetermined period arrives).

Here, the “automatic medical interview terminal” may be a terminal installed in a medical institution or an information processing terminal (for example, a smartphone or the like) carried by a patient. Further, the symptom appearing exercise information acquisition means may receive an input of the patient information via a user interface provided by an application executed in the automatic medical interview terminal. Further, the application may include a daily health management function. This can also serve as a health management for the patient.

When an interval of inputs between two or more inputs of the symptom appearing exercise information performed by the patient exceeds the first predetermined period, the medical care assistance image generation means may display the estimated severity display bar with a length of the first predetermined period backward from a timing at which a subsequent input of the symptom appearing exercise information is performed.

In a case where the interval of the information input (hereinafter, also referred to as an answer) of the patient exceeds the first predetermined period from the time of the previous answer, by performing the display of the estimated severity display bar as described above, a blank period (a period in which the bar is not displayed on the graph) occurs from the time of the previous answer. This makes it easy for the physician and the patient to commonly recognize that “the evaluation is performed every first predetermined period”. In addition, it is possible to motivate the patient to periodically perform information input not making a blank period to occur as much as possible. In addition, when the bar is excessively extended in the past, there is a risk that severity different from the actual severity is displayed in a case where there is a change in the disease condition within the period. However, it is possible to prevent the risk by setting a limit of the length of the bar to the first predetermined period.

When an interval of inputs between two or more inputs of the symptom appearing exercise information performed by the patient is shorter than the first predetermined period, the medical care assistance image generation means may set the interval as the alternative first predetermined period, and display the estimated severity display bar with a length of the alternative first predetermined period backward from a timing at which a subsequent input of the symptom appearing exercise information is performed.

If the length of the estimated severity display bar based on the most recent answer is set to the same length as the first predetermined period in a case where the interval of the information input of the patient does not reach the first predetermined period from the time of the previous answer, the estimated severity display bar based on the answer content at the time of the previous answer is overwritten (the bar extends beyond the date of the previous answer date). Therefore, when the interval between the answers is shorter than the first predetermined period, the period corresponding to the interval between the answers is set as the alternative first predetermined period as described above, and the length of the estimated severity display bar is set as the length of the alternative first predetermined period, whereby such overwriting can be prevented, and the severity of the patient can be comprehended more accurately.

The medical care assistance image generation means may generate the medical care assistance image in that the estimated severity display bar is displayed in color-coded manner according to the estimated severity information and text information related to the minimum exercise intensity and/or the estimated severity information of the patient in the first predetermined period is indicated by superimposing on the estimated severity display bar or in the vicinity of the estimated severity display bar. According to such a configuration, the physician can check the transition of the estimated severity more easily by referring to the medical care assistance image.

The disease of medical care target may be heart failure, and the estimated severity information may be assumed to be NYHA classification. The present invention is suitable for such a case.

The present invention can also be regarded as a medical care assistance device including, the minimum exercise intensity calculation means, the estimated severity information calculation means, and the medical care assistance image generation means, the medical care assistance device constituting at least a part of the medical care assistance system.

Further, the present invention can also be considered as a program for causing a computer to function as such a medical care assistance device, and a computer-readable recording medium recording such a program in a non-transitory manner.

Also, the configurations and processing described above can be combined with one another to constitute the present invention unless the combination leads to contradiction.

### Advantageous Effects of Invention

According to the present invention, it is possible to provide a technique for a system related to medical assistance, which is capable of reducing a workload on a medical professional to perform diagnosis including diagnosis about severity of a disease of medical care target for a patient.

### BRIEF DESCRIPTION OF DRAWINGS

FIG. 1 is a schematic diagram illustrating a configuration of a medical care assistance system according to an example.

FIG. 2 is a block diagram illustrating a functional configuration of a server device according to the example.

FIG. 3 is a diagram illustrating an example of a data table according to the example.

FIG. 4 is a block diagram illustrating a functional configuration of a physician-side terminal according to the example.

FIG. 5 is a first diagram illustrating an example of a medical care assistance image output on a physician-side terminal.

FIG. 6A is a second diagram illustrating an example of the medical care assistance image output on the physician-side terminal. FIG. 6B is a third diagram illustrating an example of the medical care assistance image output on the physician-side terminal.

FIG. 7A is a fourth diagram illustrating an example of the medical care assistance image output on the physician-side terminal. FIG. 7B is a fifth diagram illustrating an example of the medical care assistance image output on the physician-side terminal.

FIG. 8A is a sixth diagram illustrating an example of the medical care assistance image output on the physician-side terminal. FIG. 8B is a seventh diagram illustrating an example of the medical care assistance image output on the physician-side terminal.

FIG. 9 is an eighth diagram illustrating an example of the medical care assistance image output on the physician-side terminal.

FIG. 10 is a block diagram illustrating a functional configuration of a patient-side terminal according to the example.

FIG. 11A is a first diagram illustrating an example of a user interface displayed on the patient-side terminal. FIG. 11B is a second diagram illustrating an example of the user interface displayed on the patient-side terminal.

FIG. 12 is a diagram illustrating a flow of information exchange and processing performed in the medical care assistance system according to the example.

FIG. 13 is a diagram illustrating an outline of a medical care assistance system according to another aspect.

### DESCRIPTION OF EMBODIMENTS

### Example 1

Embodiments of the present invention will be specifically described below with reference to the drawings. However, it should be noted that the dimension, shape, relative arrangement and the like of the components described in this embodiment are not intended to limit the scope of this invention to them alone, unless otherwise stated.

### System Configuration

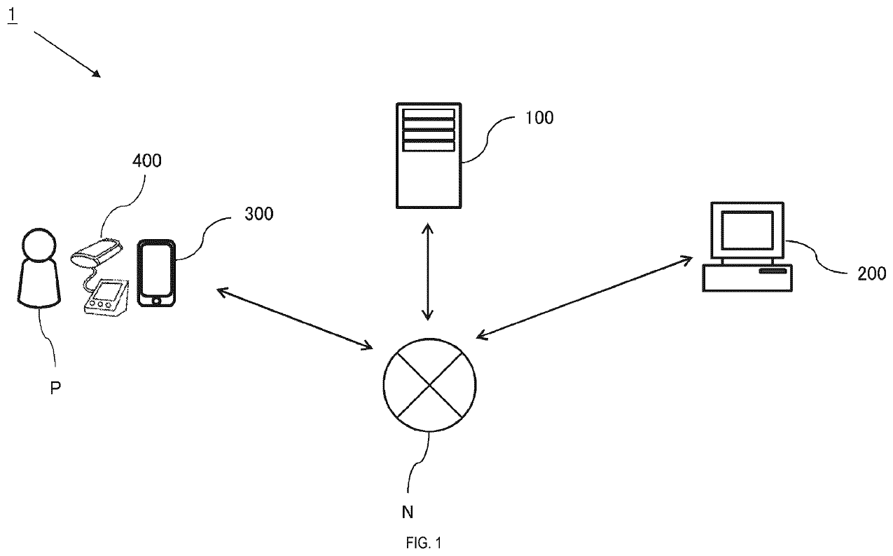

FIG. 1 is a schematic diagram illustrating a configuration of a medical care assistance system 1 according to the present example. As illustrated in FIG. 1, the medical care assistance system 1 is configured by including a server device 100, a physician-side terminal 200 used by a physician, and a patient-side terminal 300 and a measuring device 400 that are used by a patient P. All of these in the system configuration can communicate with one another via a communication network N.

The medical care assistance system 1 according to the present example is a system related to medical care, which assists a physician to perform treatment on a patient by transmitting measurement values of biological information such as a heart rate, a pulse rate, a blood pressure value, and a weight measured by the patient at home to the server device 100 via the communication network N, processing the information, and providing the information to a medical professional.

A patient who is determined that he or she needs continuous monitoring of biological information by receiving, for example, a definitive diagnosis of the heart failure or the like, starts treatment in accordance with a diagnosis by the physician, continuously measures biological information at home by himself or by herself, and records subjective symptoms in daily life. The medical care assistance system 1 collects information related to the measured value and the subjective symptom, generates a medical care assistance image for a medical professional such as a physician to refer to regarding medical care of the patient based on the information being collected, and outputs the medical care assistance image via an output means. The medical care assistance image is referred to at the time of medical examination of a patient and is appropriately referred to as necessary for medical care.

The medical care assistance system 1 may indicate alert information of the medical care assistance image when the collected measurement value of the patient satisfies an alert condition set in advance. Further, the alert signal may be transmitted to an information processing terminal, a portable communication terminal, or the like possessed by the physician. Hereinafter, the respective configurations of the system will be described in detail.

### Server Device

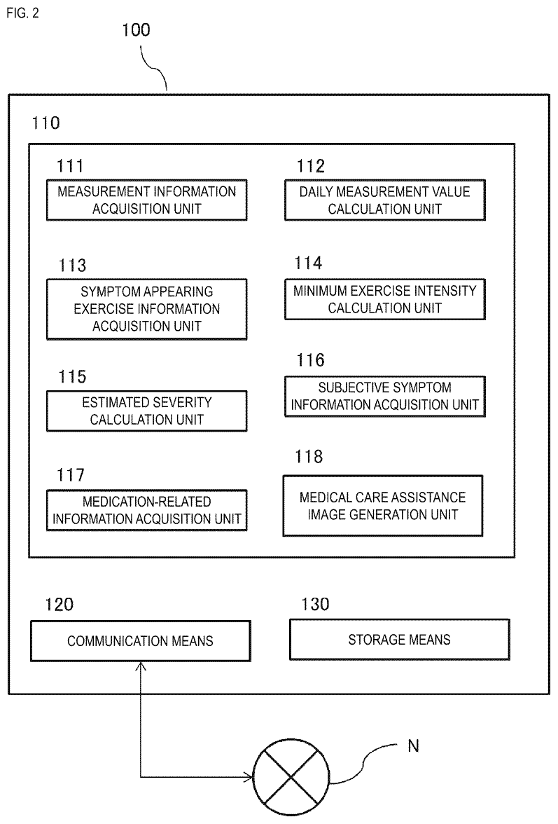

FIG. 2 is a block diagram illustrating a functional configuration of the server device 100. The server device 100 is constituted of a general server computer and, as illustrated in FIG. 2, includes a control unit 110, a communication means 120, and a storage means 130.

The control unit 110 is a means that manages the control of the server device 100, and is constituted of a processor, such as a central processing unit (CPU) or a digital signal processor (DSP). The control unit 110 includes, as functional modules related to biological information management, functional units such as a measurement information acquisition unit 111, a daily measurement value calculation unit 112, a symptom appearing exercise information acquisition unit 113, a minimum exercise intensity calculation unit 114, an estimated severity calculation unit 115, a subjective symptom information acquisition unit 116, a medication-related information acquisition unit 117, and a medical care assistance image generation unit 118. These respective functional units will be described in detail below.

The communication means 120 is a communication means configured to connect the server device 100 to the communication network N, and is constituted including, for example, a communication interface board and a wireless communication circuit for wireless communication.

Although not illustrated, the storage means 130 includes a main storage unit, such as a read only memory (ROM) or a random access memory (RAM), and an auxiliary storage unit, such as an EPROM, a hard disk drive (HDD), a solid state device (SSD), or a removable medium. The auxiliary storage unit stores an operating system (OS), various programs, and the like. By the stored programs being loaded into a work area of the main storage unit and executed, and the respective component units and the like being controlled by the execution of the programs, the respective functional units that meet predetermined purposes can be realized.

As will be described below, the measurement information acquisition unit 111 acquires, via the communication network N, measurement values of biological information such as a heart rate, a pulse rate, a blood pressure value, and a weight measured by the patient P with the measuring device 400, and stores the measurement values in the storage means 130. These measurement values can be acquired by various known measuring devices. Further, as the measuring device, separate devices corresponding to each biological information may be used, or a measuring device that can acquire different measurement values by one device (one measurement), for example, an upper arm-type oscillometric blood pressure monitor that can acquire a blood pressure value and a pulse rate may be used.

In addition, in a case where a specific symptom such as atrial fibrillation (AF) or a suspicion thereof is detected at the time of heart rate measurement, the measurement information acquisition unit 111 acquires information of the specific symptom or the suspicion together with the heart rate and stores the information in the storage means 130. In addition, when a specific symptom such as arrhythmia or a suspicion thereof is measured at the time of pulse rate measurement, the measurement information acquisition unit 111 also acquires information indicating measurement of the specific symptom or the suspicion and stores the information in the storage means 130. The information of the measurement value acquired by the measurement information acquisition unit 111 includes time information at which the measurement is performed and information about a place at which the measurement is performed (for example, home or medical examination room).

The daily measurement value calculation unit 112 calculates one heart rate measurement value and one pulse rate measurement value in one opportunity of the patient P for each day based on the measurement values stored in the storage means 130 and a predetermined calculation rule, and stores the calculated values in the storage means 130. The term “one opportunity” refers to a measurement timing for one piece of biological information, such as “morning (within one hour after getting up)” and “evening (before going to bed)” in the guidelines for diagnosis and treatment of high blood pressure. In the present example, with respect to such a measurement timing, for example, a certain constant time span is set as “one opportunity=10 minutes (from the start of the measurement of the first biological information)”, and a series of a plurality of pieces of biological information (including different kinds of biological information) measured within the time span are collectively set as “biological information obtained in one opportunity”. That is, regarding the measurement of the biological information, when the measurement is performed a plurality of times within the certain constant period of time, the plurality of measurements is collectively set as the measurement of one opportunity, and the pieces of biological information measured a plurality of times correspond to the biological information obtained in one opportunity. Here, in any of a case where only the identical biological information is measured a plurality of times, a case where different pieces of biological information are each measured once, and a case where different pieces of biological information are each measured once or more, the plurality of measurements corresponds to the measurement in one opportunity if all the measurements are performed within a certain constant period of time.

On the other hand, even when one piece of biological information is measured twice, if the two measurements are not performed within a certain constant period of time (for example, once at the time of getting up in the morning and once at the time before going to bed at night), the two pieces of biological information correspond to the biological information measured at different opportunities (related to the measurement of two opportunities).

Here, the calculation of the daily measurement value by the daily measurement value calculation unit 112 will be specifically described using the heart rate as an example. First, in a case where the heart rate is measured only once a day and only the measurement value is stored in the storage means 130, the daily measurement value calculation unit 112 sets the measurement value as the daily heart rate measurement value. On the other hand, when a plurality of measurements is performed in one day and all of the plurality of measurements are within a predetermined time, the daily measurement value calculation unit 112 determines that all of the plurality of measurement values are measurement values within one opportunity, obtains one value (for example, an average value of the plurality of measurement values) as a measurement value in one opportunity based on the plurality of measurement values, and calculates the value as a daily heart rate measurement value. In addition, in a case where a plurality of measurements are performed in one day and the plurality of measurements are not within a predetermined time (that is, in a case where measurements for a plurality of opportunities are performed), the daily measurement value calculation unit 112 calculates a daily heart rate measurement value using a measurement value of any one opportunity among the plurality of opportunities (for example, a measurement value of a measurement opportunity at a timing set in advance such as a measurement value of one opportunity at the time of getting up in the morning). In this case, the method of obtaining the measurement value of one opportunity when a plurality of measurements is performed within the one opportunity is as described above. Here, the heart rate is described as an example, but the daily measurement value calculation unit 112 performs the similar calculation processing for other biological information such as a pulse rate.

The symptom appearing exercise information acquisition unit 113 acquires symptom appearing exercise information which is information including exercise contents in which a symptom related to a disease of medical care target for the patient P (here, heart failure) appears, and stores the symptom appearing exercise information in the storage means 130. Specifically, by causing the patient P to input or select exercise (physical activity) in which the patient P is aware of the symptom every predetermined period (for example, one week) by an application executed in the patient-side terminal 300 to be described below, the symptom appearing exercise information is acquired via the patient-side terminal 300.

The minimum exercise intensity calculation unit 114 calculates the minimum exercise intensity, which is the exercise intensity of the exercise having the smallest exercise intensity among the exercises in which the symptom related to heart failure has appeared within a predetermined period, based on the symptom appearing exercise information stored in the storage means 130. In the present example, the exercise intensity is indicated by the METs, and hereinafter, the exercise intensity of the exercise having the smallest exercise intensity among the exercises in which the symptom related to heart failure has appeared within a predetermined period is also referred to as a symptom appearance minimum METs. Specifically, the minimum exercise intensity calculation unit 114 may hold, in the storage means 130, an exercise intensity table in which the content of exercise and the exercise intensity of the exercise are associated with each other, and may obtain the symptom appearance minimum METs by referring to the exercise intensity table. The symptom appearance minimum METs calculated here is stored in the storage means 130.

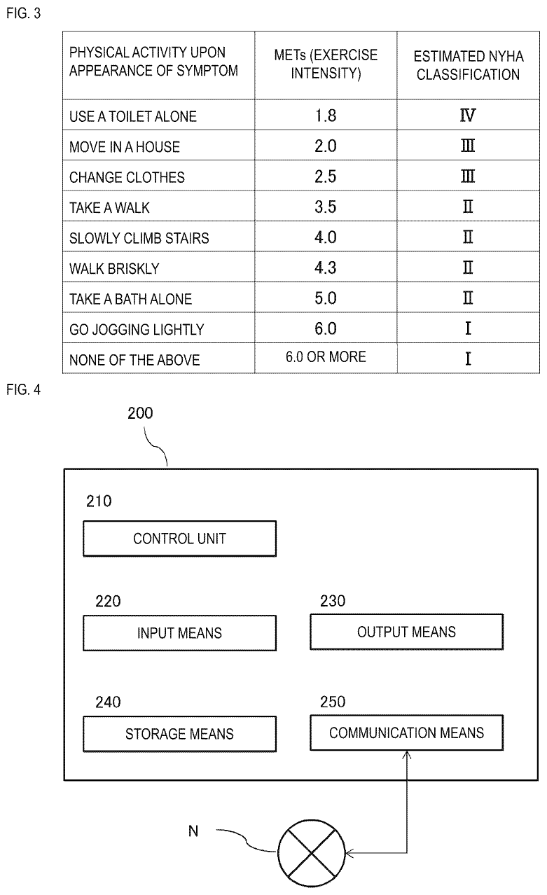

The estimated severity calculation unit 115 obtains estimated severity information indicating the severity of heart failure based on the symptom appearance minimum METs stored in the storage means 130. In the present example, the severity is based on the NYHA classification, and hereinafter, the estimated severity is also referred to as an estimated NYHA classification. For example, the estimated severity calculation unit 115 may obtain the estimated severity by referring to a data table stored in the storage means 130 in which the symptom appearance minimum METs number and the estimated NYHA classification are associated with each other. In FIG. 3, an example of a data table is illustrated in which the exercise contents, the METs (numerical values) of the exercise, and the estimated NYHA classification in the case where the METs of the exercise is the symptom appearance minimum METs number are associated with each other. Note that the exercise contents (and the corresponding METs and estimated NYHA classifications) illustrated in FIG. 3 are representative excerpts, and more exercise contents are actually stored. The estimated severity calculated here is stored in the storage means 130.

The subjective symptom information acquisition unit 116 acquires information of the presence or absence (and the type) of a symptom related to heart failure for each predetermined period (for example, every day) in the patient P, and stores the information in the storage means 130. Specifically, the information is acquired via the patient-side terminal 300 by causing the patient P to select a symptom that the patient P is aware of at a fixed time every day by an application executed in the patient-side terminal 300, similarly to the symptom appearing exercise information. Specifically, for example, a list of symptoms may be presented to allow the patient to select a symptom from the list, or input of text information related to a subjective symptom may be received as a memo or the like.

In addition, the medication-related information acquisition unit 117 acquires information regarding the presence or absence of medication and the medication rate (frequency of medication) of the patient, and stores the information in the storage means 130. In addition, information regarding the contents, frequency, and the like of side effects at the time of medication may be acquired. These pieces of information may be acquired via the patient-side terminal 300, for example, by causing the patient P to select the presence or absence of the medication of the day at a fixed time every day by the application executed in the patient-side terminal 300, similarly to the symptom appearing exercise information. Further, the medication-related information acquisition unit 117 may cooperate with an external system (for example, an electronic medical chart system) or the like, which is not illustrated, to acquire information (prescription information) of medication prescribed to the patient P.

The medical care assistance image generation unit 118 generates a medical care assistance image to be referred by a medical professional based on data output from each functional unit of the measurement information acquisition unit 111, the daily measurement value calculation unit 112, the symptom appearing exercise information acquisition unit 113, the minimum exercise intensity calculation unit 114, an estimated severity calculation unit 115, the subjective symptom information acquisition unit 116, and the medication-related information acquisition unit 117, and stored in the storage means 130. The generated medical care assistance image is transmitted to the physician-side terminal 200 via the communication network N. The medical care assistance image will be described in detail later.

### Physician-Side Terminal

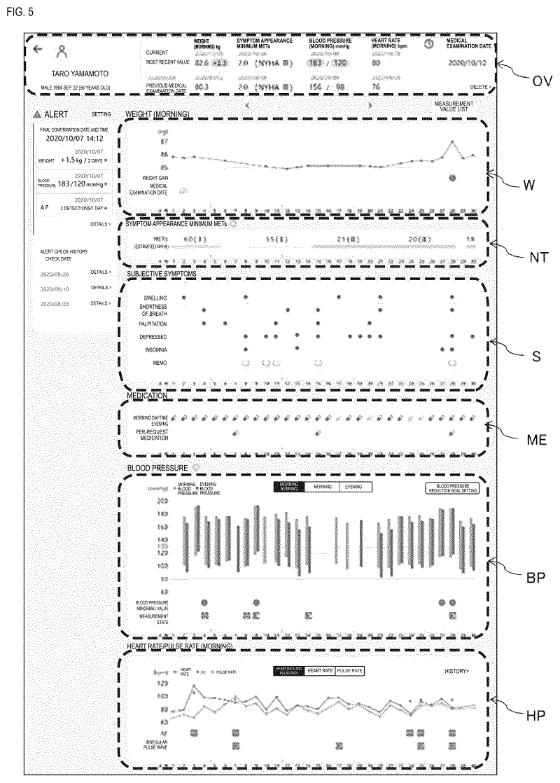

FIG. 4 is a block diagram illustrating a functional configuration of the physician-side terminal 200. The physician-side terminal 200 is a general computer, such as a fixed installation type personal computer, a portable notebook-type personal computer, or a tablet type terminal, and includes a control unit 210, an input means 220, an output means 230, a storage means 240, and a communication means 250.

The control unit 210 is a means that controls the physician-side terminal 200, and is constituted of, for example, a CPU. The input means 220 is a means that receives information input from the outside, including such as for example, a keyboard, a mouse, a touch panel, a camera, or a microphone. The output means 230 includes a liquid crystal display, a speaker, a printer, and the like. The storage means 240 is constituted similarly to the server device by including a main storage unit, an auxiliary storage unit, and the like, and stores various data, for example, an operating system (OS), various programs, and the like acquired via the communication network N. The communication means 250 is constituted including, for example, a communication interface board and a wireless communication circuit for wireless communication.

Note that, although not illustrated, the physician-side terminal may be accessible to an electronic medical chart management system. In such a case, electronic medical chart data of a patient stored in the electronic medical chart management system may be read out and the electronic medical chart data may be transmitted to the server device 100, or information transmitted from the server device 100 may be associated with the electronic medical chart data. In such a case, the physician can check the medical care assistance image via the electronic medical chart management system.

In the physician-side terminal 200, the medical care assistance image is acquired from the server device 100 via the communication network N, and such information is output to the output means 230. An example of screen (medical care assistance image) displayed on the output means 230 of the physician-side terminal 200 is illustrated in FIG. 5 to FIG. 9. FIG. 5 is an explanatory diagram illustrating an example of a medical care assistance image for one of the patients P managed by the physician who is the administrator of the terminal. As illustrated in FIG. 5, the medical care assistance image according to the present example is configured to include a plurality of regions indicating different information. Specifically, a summary information region OV, a weight information region W, an estimated NYHA classification transition region NT, a subjective symptom information region S, a medication information region ME, a blood pressure information region BP, and a heart rate/pulse rate information region HP are included. Note that the entire medical care assistance image need not be displayed on the output means, and a display region can be selected (scrolled, reduced, or enlarged) as appropriate. In addition, the medical care assistance image may be generated in a combination and order of items designated by the physician in advance.

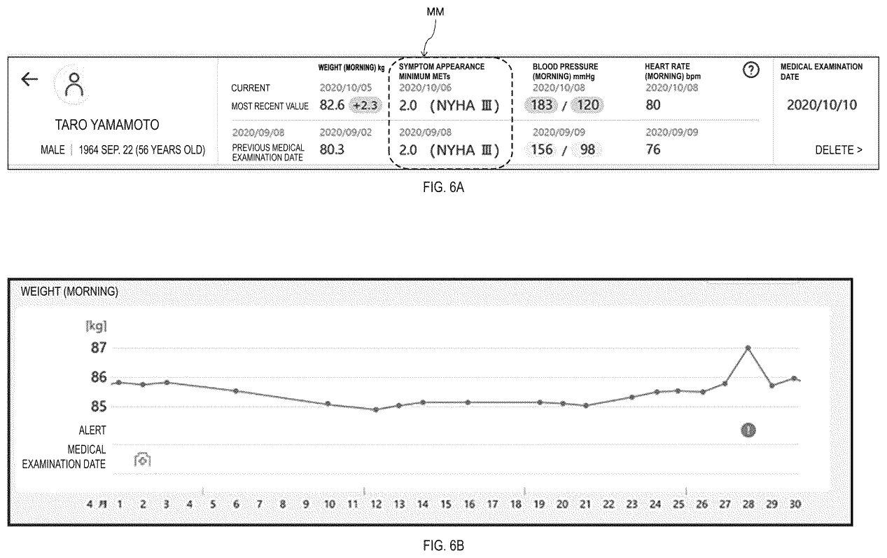

Hereinafter, information displayed in each region of the medical care assistance image will be specifically described. FIG. 6A is an enlarged view of the summary information region OV. As illustrated in FIG. 6A, the summary information region OV displays information regarding patient attributes such as patient name, gender, and age, patient information acquired most recently, and patient information at the time of the previous medical examination. In addition, Minimum METs information MM indicating the symptom appearance minimum METs number (and the estimated NYHA classification) is illustrated as one piece of patient information. By such a display, the physician can check the most recent symptom appearance minimum METs (and the estimated NYHA classification) of the patient P and refer to the previous symptom appearance minimum METs (and the estimated NYHA classification), and can efficiently perform a medical interview for diagnosing the severity of the patient P at the time of medical examination.

FIG. 6B is an enlarged view of the weight information region W. As illustrated in FIG. 6B, in the weight information region W, the transition of the weight of the patient P during the display period (for example, from the first day to the last day of the previous month, the past one month, the past one week, or the like) is illustrated by a graph. When the function of the heart deteriorates, water tends to accumulate in the body due to poor blood flow. Therefore, an increase in body weight (for example, an amount of increase per week) (derived from the accumulated water) is an important index related to the severity of heart failure. For this reason, alert information may be displayed in the weight information region W in a case where an increase or decrease value of the weight within a predetermined period deviates from a threshold value.

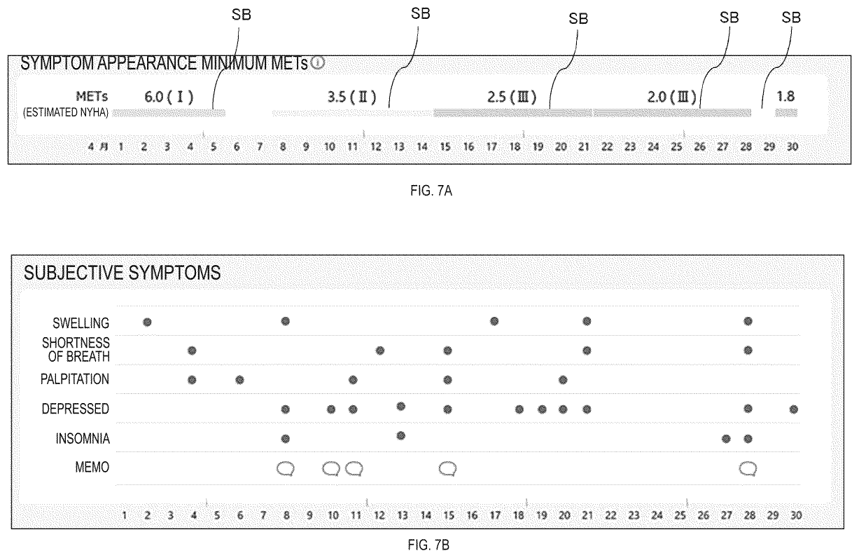

FIG. 7A is an enlarged view of the estimated NYHA classification 10 transition region NT. As illustrated in FIG. 7A, in the estimated NYHA classification transition region NT, an estimated severity time series graph is illustrated in which the estimated NYHA classification for each predetermined period within the display period (30 days) is indicated by an estimated severity display bar SB in which the difference in class can be identified by the difference in color. In addition, in the vicinity of the estimated severity display bar SB, the corresponding symptom appearance minimum METs (and the estimated NYHA classification) are displayed in text. By such a display, the physician can easily check the transition of the estimated NYHA classification of the patient within the display period, and can efficiently perform the medical interview for diagnosing the severity of the patient P at the time of medical examination.

Since the estimated severity display bar SB displays the estimated NYHA classification calculated based on the symptom appearing exercise information acquired (that is, input by the patient) by the symptom appearing exercise information acquisition unit 113 for each predetermined period, the estimated severity display bar SB is basically displayed for each predetermined period (for example, one week) according to the information acquisition interval. However, the estimated severity display bar SB may be displayed in a period shorter than the predetermined period, for example, when the acquisition timing of the information by the symptom appearing exercise information acquisition unit 113 is changed or when the predetermined period includes a schedule before the first day or after the last day of the display region. Such a period corresponds to an alternative first predetermined period according to the present invention. Further, the estimated severity display bar SB may be displayed with a length of a period exceeding a predetermined period. The period in such a case also corresponds to the alternative first predetermined period. The length of the minimum period in which the estimated severity display bar SB is displayed is one day.

Here, the length (period in the graph) of the estimated severity display bar SB can be determined by the medical care assistance system 1, for example, as follows. In a case where the acquisition interval of the symptom appearing exercise information by the symptom appearing exercise information acquisition unit 113 (that is, the interval of the input of the information by the patient) is in accordance with the first predetermined period, the medical care assistance system 1 determines the length of the estimated severity display bar SB as a length that is backward by a predetermined period from the time of the answer. The third bar from the left in FIG. 7A corresponds to such a case, and when the timing of the answer is the 21st day of the display period (30 days), a length of the estimated severity display bar SB is determined as a length that is backward by the first predetermined period (7 days) from the time of the answer (21st day), that is, 7 days from the 15th day to the 21st day. Then, the medical care assistance image generation unit 118 creates a medical care assistance image in which the estimated severity display bar SB reflecting the determined length (the number of days) is displayed.

On the other hand, in a case where the answer interval of the patient exceeds the first predetermined period, the medical care assistance system 1 determines the length of the estimated severity display bar SB as a length that is backward by the first predetermined period (7 days) from the time of the subsequent answer. The second estimated severity display bar SB from the left in FIG. 7A corresponds to such a case, and when the timing of the answer is the 14th day, a length of the estimated severity display bar SB is determined as a length that is backward by the first predetermined period (7 days) from the time of the answer (14th day), that is 7 days from the 8th day to the 14th day. Then, the medical care assistance image generation unit 118 creates a medical care assistance image in which the estimated severity display bar SB reflecting the determined length (the number of days) is displayed.

In a case where the answer interval of the patient is shorter than the predetermined period, the medical care assistance system 1 determines the answer interval as the alternative first predetermined period, and determines the length of the estimated severity display bar SB as a length that is backward by the alternative first predetermined period from the time of the subsequent answer. The second estimated severity display bar SB from the right in FIG. 7A corresponds to such a case, and when the interval from the previous answer date (21st day) to the subsequent answer date (27th day) is six days (=shorter than the first predetermined period), a length of the alternative first predetermined period is determined to be six days. Then, the medical care assistance image generation unit 118 creates a medical care assistance image in which the estimated severity display bar SB reflecting the determined length (the number of days) of the alternative first predetermined period is displayed. In this manner, it is possible to prevent the estimated severity display bar SB (the third bar from the left) of the previous first predetermined period from being overwritten. The same applies to the determination method and the display method of the alternative first predetermined period in a case where the acquisition interval of the symptom appearing exercise information is in accordance with the first predetermined period while a schedule before the first day or after the last day of the display region is included (the estimated severity display bars SB at both ends of the FIG. 7A correspond to this case).

FIG. 7B is an enlarged view of the subjective symptom information region S. As illustrated in FIG. 7B, in the subjective symptom information region S, information is displayed indicating whether or not there is a subjective symptom related to heart failure by displaying dots for each type of symptom for each day along a time series (a symptom displayed by dots is a symptom that is perceived on that day). In addition, in a case where the patient writes a daily memo via the patient-side terminal 300 described below, a display indicating this may also be displayed. By referring to such a display, the physician can easily check the transition of what kind of subjective symptom is felt by the patient P every day (the kind and the frequency).

In addition, by arranging the subjective symptom information region S and the estimated severity display bar SB side by side aligning on the same time axis, the physician can easily check the correspondence relationship between the daily transition of the subjective symptom and the transition of the estimated severity, and can efficiently comprehend the progress of the disease condition of the patient.

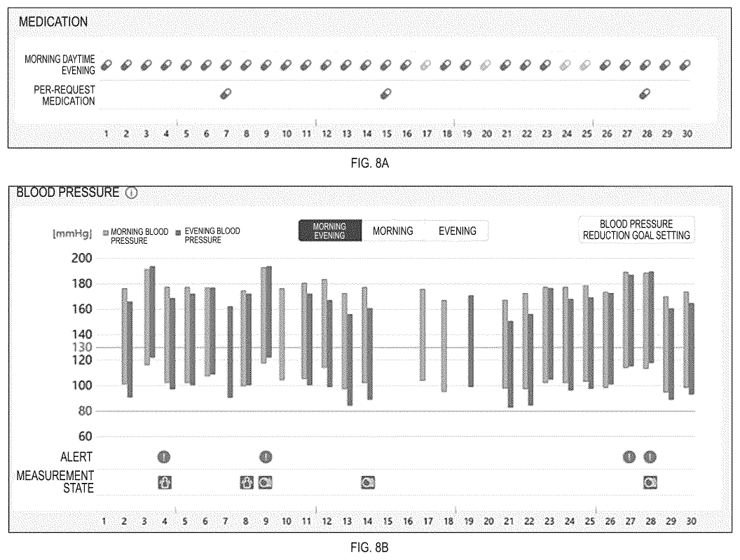

FIG. 8A is an enlarged view of the medication information region ME. As illustrated in FIG. 8A, in the medication information region ME, information of the medication of the patient within the display period (whether or not the patient has correctly taken the prescribed medication) is indicated on a daily basis by activation/deactivation of display of the capsule mark. In addition, in a case where the patient takes the per-request medication, the fact is separately displayed in the column of the date of taking the medication. In addition, when the medication is to be taken a plurality of times per day (for example, in the morning, daytime, and in the evening), a column of the presence or absence of taking the medication corresponding to each time may be provided. Alternatively, a display related to the dosing adherence rate per day (for example, a display of marks corresponding to the number of taken medications, a display of fractions, or the like) or a display using a pie chart may be performed.

FIG. 8B is an enlarged view of the blood pressure information region BP. As illustrated in FIG. 8B, in the blood pressure information region BP, the blood pressure value in the display period is displayed on a daily basis. Specifically, the blood pressure value of one opportunity is illustrated by a bar graph in which the systolic blood pressure is an upper end and the diastolic blood pressure is a lower end. When there are measurement values of two or more opportunities (for example, at the time of getting up in the morning and at the time of going to bed at night), these measurement values can be displayed in parallel as illustrated in FIG. 8B. In addition, the measurement opportunity (for example, morning/evening/others) can be displayed being identifiable by color-coded display or the like.

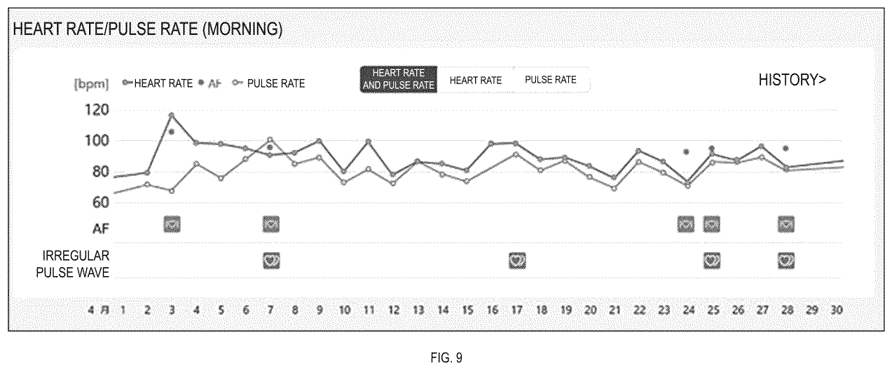

FIG. 9 is an enlarged view of the heart rate/pulse rate information region HP. As illustrated in FIG. 9, in the heart rate/pulse rate information region HP, a graph is displayed in which the daily heart rate and the daily pulse rate of each day calculated by the daily measurement value calculation unit 112, and the heart rate at the time when atrial fibrillation (AF) is detected in a case where the AF is detected are plotted on the same graph area (the X axis is the time axis, and the Y axis is the number of beats). In the graph, the daily heart rate and the daily pulse rate are one numerical value per day. However, as for the heart rate at the time when the AF is detected, in a case where the AF is detected a plurality of times per day, all of the heart rates when the AF is detected are plotted. In this manner, it is possible to distinguish between an item for which a change is desired to be tracked in time series and an item for which single information is desired to be comprehended, and to comprehend them in association with each other. Further, on a day when the AF is detected and on a day when an irregular pulse wave (arrhythmia) is detected, marks indicating the detection may be separately displayed.

In the example illustrated in FIG. 9, there is a measurement opportunity in which the daily measurement values of the heart rate and the pulse rate can be determined on all days of the display period, and the heart rate and the pulse rate of the measurement opportunity are plotted. On the other hand, when there is a day without a measurement opportunity in which both the heart rate and the pulse rate are appropriately measured, both the heart rate and the pulse rate may not be plotted for the day. Alternatively, a biological information value to be prioritized among the heart rate and the pulse rate may be determined in advance, and only the prioritized biological information value may be plotted (displayed). Alternatively, only the biological information value that is appropriately measured may be displayed in a mode in which the value can be identified as a reference value.

By referring to such a display, the physician can easily check the transition of the systole function of the patient P. In addition, by plotting the heart rate and the pulse rate in the same graph area, even if there is a measurement error in either the measurement of the heart rate or the measurement of the pulse rate, the systole function of the patient P can be diagnosed by the other numerical value. In a case where there is a difference between the heart rate and the pulse rate, it is possible to examine and determine, by considering other information, whether the difference is derived from a measurement error or an event to be noted such as a change in the symptom of the patient occurs.

The physician can efficiently acquire the information regarding the patient P by referring to the medical care assistance image on which the information as described above is displayed, and the burden on the physician who has to comprehend the information of many patients in charge can be greatly reduced. In addition, the physician refers to the medical care assistance image to eliminate futile questions from the contents of the medical interview at the time of the medical examination, and thus it is possible to reduce the burden on the patient P at the time of the medical examination.

### Patient-Side Terminal

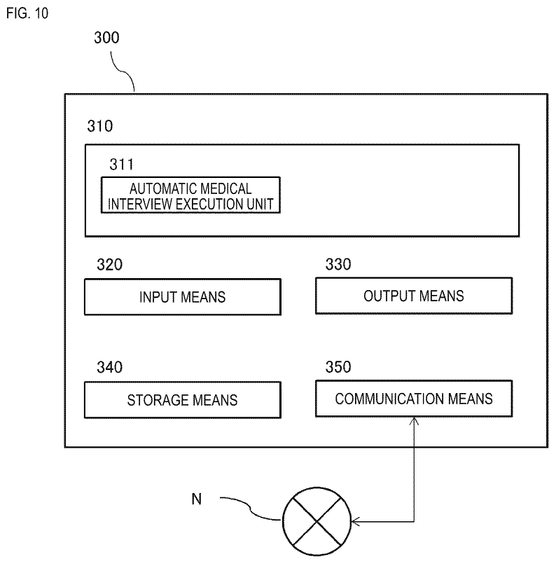

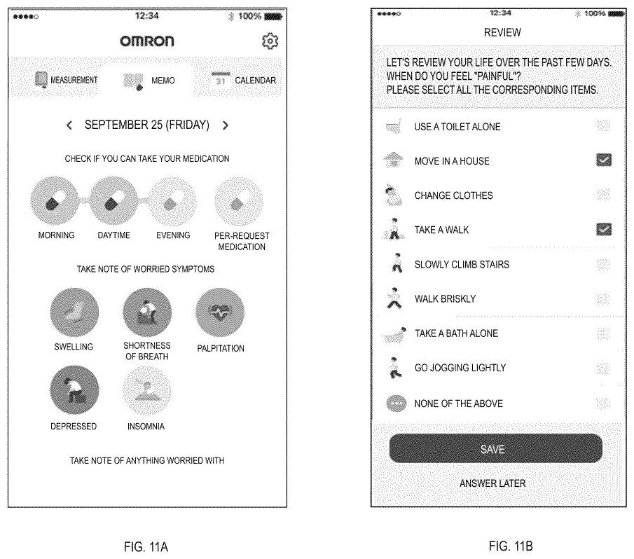

FIG. 10 is a block diagram illustrating a functional configuration of the patient-side terminal 300. The patient-side terminal 300 is, for example, a portable information processing terminal, such as a smartphone, a tablet terminal, or a wristwatch type wearable terminal, and includes a control unit 310, an input means 320, an output means 330, a storage means 340, and a communication means 350. In this example, the patient-side terminal 300 corresponds to an automatic medical interview terminal according to the present invention.

The control unit 310 is a means for controlling the patient-side terminal 300, and is constituted of, for example, a CPU and the like. As the input means 320, a touch panel display integrated with the output means 330 or the like can be adopted. The storage means 340 is constituted similarly to other terminals by including a main storage unit, an auxiliary storage unit, and the like, and stores various data, for example, an operating system (OS), various programs, and the like acquired via the communication network N. The communication means 250 is constituted including, for example, a wireless communication circuit for wireless communication.

The control unit 310 includes an automatic medical interview execution unit 311 as a functional module related to patient information management including symptom appearing exercise information and the like. The automatic medical interview execution unit 311 is implemented, for example, as a function provided by an application program, and receives an input of patient information via a user interface (hereinafter, referred to as UI) for requesting a user to input information to perform a medical interview. The automatic medical interview execution unit 311 may display, for example, a UI that displays a plurality of icons related to predetermined items and prompts the user to make a selection, or may adopt a format such as a so-called chatbot. Further, the application program may be stored in the storage means 340 of the patient-side terminal 300, or may be provided in the form of software as a service (SaaS) in the server device 100.

The automatic medical interview execution unit 311 executes an automatic medical interview for each predetermined period set according to the information (for example, medication information, information of the presence or absence of a subjective symptom, symptom exercise information, and the like) that is requested to the patient to input. In addition, notification (screen display, voice output, or the like) for prompting the patient to input information is performed at a timing at which the automatic medical interview is executed (that is, every predetermined period).

FIG. 11A and FIG. 11B are diagrams illustrating an example of a state in which a UI provided by the automatic medical interview execution unit 311 is displayed on a screen of a smartphone as an example of the patient-side terminal 300. In FIG. 11A, a UI is illustrated which receives an input of medication information and information of the presence or absence of a subjective symptom (subjective symptom information) for each day. As illustrated in FIG. 11A, the UI is configured such that the medication information is input by selecting a medication icon for each time zone of morning, daytime, and evening, and display of the selected icon is activated. In addition, as for the subjective symptom information, the UI is also configured such that the icons indicating the respective symptoms are displayed, and an input is performed by selecting the icon of the subjective symptom. Again, the selected icon is activated for display. The automatic medical interview execution unit 311 executes an automatic medical interview processing of requesting the patient to input the medication information and the subjective symptom information at a scheduled time (for example, 21:00) every day through the screen of FIG. 11A. Note that the screen illustrated in FIG. 11A is an example of the UI related to an input of the medication information and the subjective symptom, and the user may be requested to input the subjective symptom by another UI. Specifically, for example, a list of symptoms may be presented to allow the patient to select a symptom from the list, or input of text information related to a subjective symptom may be received as a memo or the like.

In FIG. 11B, an example of a UI is illustrated which receives an input of symptom appearing exercise information for each predetermined period (for example, one week). As illustrated in FIG. 11B, the UI is configured such that a list of items indicating a plurality of exercise contents (physical activities) with different exercise intensities are displayed, and an input is performed by selecting the physical activity in which a subjective symptom appears. A check mark is displayed for the selected physical activity, and thus the selected item is clearly illustrated. The screen illustrated in FIG. 11B is an example of the UI related to the symptom appearing exercise information input, and other UIs may be used.

The automatic medical interview execution unit 311 executes an automatic medical interview processing of requesting the patient to input symptom appearing exercise information at a timing set in advance (for example, 21:00 every Saturday) through the screen of FIG. 11B. The timing at which the automatic medical interview execution unit 311 performs the automatic medical interview processing (the timing of the notification for prompting the patient to input information) is not limited to “each specific day of the week (and time)”, that is, the timing obtained by applying a predetermined period to the calendar as described above, and may be a timing relatively calculated using the previous answer date and a predetermined period, such as “7 days after the previous automatic medical interview processing execution date (answer date)”.

In addition, when the patient does not input the information even though the automatic medical interview execution unit 311 performs the notification for prompting the input of the information, the automatic medical interview execution unit 311 may perform the notification (reminder) for prompting the input of the information again at a predetermined timing without waiting for the next predetermined period to arrive. Here, the predetermined timing may be scheduled in advance, for example, at the same time of the next day. In addition, the patient may be reminded when the patient uses the patient-side terminal 300 next time. To be specific, for example, at the time of biological information measurement by the measuring device 400 to be described below, a reminder message may be displayed together with the measurement result.

Further, after receiving the input of the patient by the automatic medical interview processing, the automatic medical interview execution unit 311 may not receive information input again until the notification of the next automatic medical interview processing is performed. According to this, it is possible to prevent the answer interval of the patient from becoming shorter than the predetermined period.

The input of information performed by the patient P via the application as described above is transmitted from the communication means 350 to the server device 100 via the communication network N. As will be described below, measurement data acquired from the measuring device 400, necessary information input by the patient P, and the like are also transmitted to the server device 100 in the same manner.

### Measuring Device

The measuring device 400 is used by the patient P for daily measurement of biological information. Here, the term “measuring device 400” is used as a concept including not only one device but also a plurality of measuring devices such as a blood pressure monitor, an electrocardiograph, and a weight scale (body composition meter). In addition, each measuring device may have any form. For example, a device of a type in which an electrocardiograph and a blood pressure monitor are integrated may be used, or a body composition meter capable of performing electrocardiographic measurement may be used. In addition, the measuring device may be a stationary device or a portable device. In addition, a wearable type device which is always attached to the patient may be included. The measuring device 400 may be integrated with the patient-side terminal 300.

Various kinds of measurement data such as a heart rate, a pulse rate, a blood pressure value, and a weight measured by using the measuring device 400 are transmitted to the patient-side terminal 300 by wired or wireless communication together with information regarding a measurement time. In the case of wireless communication, a near-field wireless data communication standard such as Bluetooth (trade name) or infrared communication can be adopted as a communication interface used between the measuring device 400 and the patient-side terminal 300.

The measuring device 400 may not include a communication means. In this case, the patient P may manually input the measurement data (and the measurement date and time information) to the patient-side terminal 300, and the information may be transmitted to the server device 100.

The patient-side terminal 300 may also have the function of the measuring device 400. For example, in a case where the patient-side terminal 300 is a wearable terminal attached to the patient P, and when a measurement function is provided in the wearable terminal, the patient-side terminal 300 can also serve as the measuring device 400. Alternatively, on the contrary, for example, the stationary measuring device 400 may have a function as an information processing terminal and also serve as the patient-side terminal 300.

### Flow of Information Processing in System

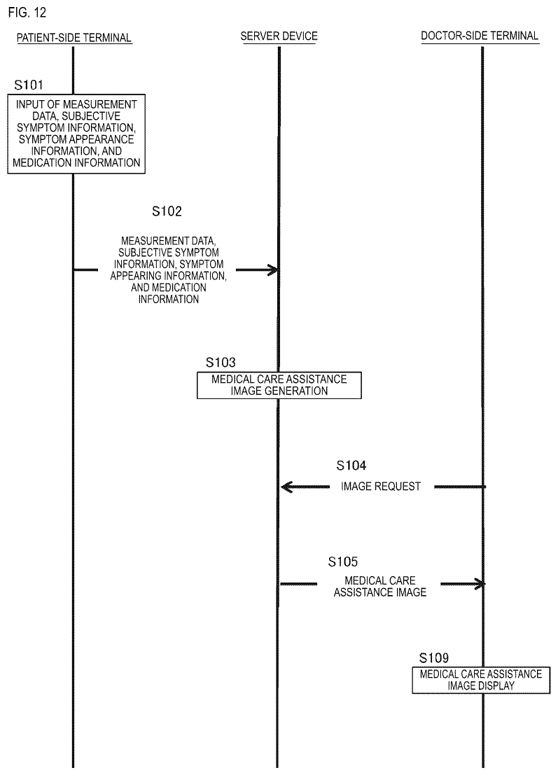

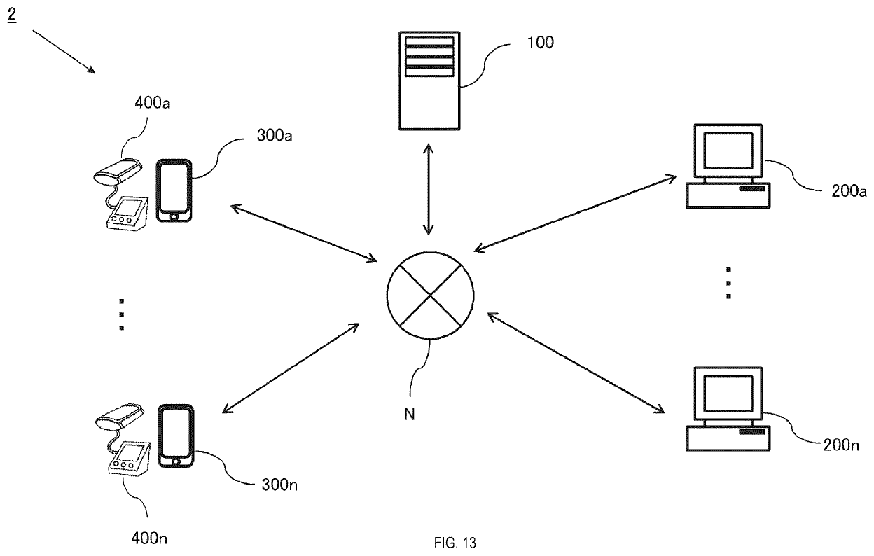

Next, a flow of information processing performed in the medical care assistance system 1 according to the present example having the above-described configuration will be described. FIG. 12 is a diagram illustrating the flow of information exchange and processing performed in the medical care assistance system 1. As illustrated in FIG. 12, first, the measurement data, the symptom appearing exercise information, the subjective symptom information, the medication information, and the like obtained by the measurement of the patient P using the measuring device 400 are input to the patient-side terminal 300 (S101). These pieces of information are transmitted from the patient-side terminal 300 to the server device 100 each time or collectively for a predetermined period (for example, one week) (S102).

In the server device 100, the received various kinds of information are stored in the storage means 130, and a medical care assistance image is generated based on the information (S103).

Thereafter, the physician transmits request information of the medical care assistance image to the server device 100 via the physician-side terminal 200 (S104). Upon receiving the request, the server device 100 provides the medical care assistance image to the physician-side terminal 200 (S105), and the medical care assistance image is displayed on the output means 230 of the physician-side terminal 200 (S106). Here, the data of the medical care assistance image may be transmitted to the physician-side terminal 200 and stored in the storage means 240 of the physician-side terminal 200, or the medical care assistance image may be provided in the form of SaaS and the image data may not be stored. The content of the medical care assistance image is as described above.

As described above, according to the medical care assistance system 1 of the present example, the physician can refer to the medical care assistance image in which the information related to the subjective symptom of the heart failure patient and the transition of the estimated severity are indicated on the time axis common to the measurement data of the biological information. According to such a screen, it is possible to efficiently and easily comprehend the transition of the disease condition and the most recent state of the patient, and it is possible to efficiently diagnose the patient by suppressing an inefficient medical interview at the time of each medical examination.

### Modified Example 1

In the above-described example, when the acquisition interval of the symptom appearing exercise information by the symptom appearing exercise information acquisition unit 113 (that is, the answer interval of the patient) exceeds the predetermined period, the estimated severity display bar SB is displayed by a length that is backward by the predetermined period from the time of the subsequent answer, but the display is not limited thereto. That is, in a case where the answer interval of the patient exceeds a predetermined period, the estimated severity display bar SB may be displayed with a length corresponding to the period from the time of the subsequent answer to the time of the previous answer. According to such a display method, a blank period in which the estimated severity display bar SB is not displayed does not occur in the estimated NYHA classification transition region NT.

### Modified Example 2

In the above-described example, the automatic medical interview execution unit 311 does not receive information input again until the notification of the next automatic medical interview processing is performed after receiving the input of the patient by the automatic medical interview processing, but this is not necessarily required. The fact that information can be input only at a predetermined timing may rather stress the patient. Therefore, even at a timing when there is no notification (that is, before the predetermined period arrives), the automatic medical interview processing may be executed to be a state where the information can be input.

Further, when the information input at the timing other than every predetermined period is received as described above, the information input by the automatic medical interview processing performed every predetermined period (that is, the regular processing) and the information input by the patient at an arbitrary timing may be provided with tagging or the like allowing identification.

In addition, in a case where a plurality of inputs of symptom appearing exercise information are received during one day, the automatic medical interview execution unit 311 may adopt (transmit to the server device 100) only information including the content of exercise when the heaviest symptom appears among the plurality of pieces of symptom appearing exercise information as the symptom appearing exercise information of the day. The minimum exercise intensity calculation unit 114 may calculate the minimum exercise intensity based on all pieces of symptom appearing exercise information after transmitting all pieces of symptom appearing exercise information to the server device 100.

### Other Points

The description of each example described above is merely illustrative of the present invention, and the present invention is not limited to the specific embodiments described above. Within the scope of the technical idea of the present invention, various modifications and combinations may be made. For example, in the above-described example, a configuration including one of each the physician-side terminal 200 and the patient-side terminal 300 has been described. However, as illustrated in FIG. 13, the present invention can be applied to a medical care assistance system 2 including a plurality of physician-side terminals 200a to 200n and/or a plurality of patient-side terminals 300a to 300n.

The medical care assistance image generation unit 118 may generate a medical care assistance image including a list indicating the contents of the data table as illustrated in FIG. 3. If it is possible to refer to the medical care assistance image including such a list at the time of medical examination, the physician can more efficiently perform a medical interview for diagnosing the severity of the patient.

In the above-described example, the automatic medical interview terminal according to the present invention has been described as the patient-side terminal 300 (the smartphone carried by the patient), but the automatic medical interview terminal is not necessarily limited thereto. For example, the automatic medical interview terminal may be an information processing terminal installed in a medical institution or the like, or may be a portable information processing terminal carried by a visiting nurse or the like and used by a patient for input.

Further, the medical care assistance system according to the present invention may not include the automatic medical interview terminal. That is, information obtained by asking a patient in a medical interview at the time of medical examination, a telephone interview, or the like may be input to the system by an operation via a mouse or a keyboard.

In the above example, the measuring device 400 transmits the measurement data to the patient-side terminal 300. However, it may be configured such that the measurement data (and information associated with the measurement data) may be directly transmitted to the server device 100. With such a configuration, even when there is no patient-side terminal 300 as an automatic medical interview terminal, the server device 100 can acquire daily measurement data of the patient P.

Further, in the above-described example, the minimum exercise intensity calculation unit 114 calculates the minimum exercise intensity based on the symptom appearing exercise information, but the minimum exercise intensity may be calculated by other methods. For example, a UI for directly inquiring about the minimum exercise intensity may be provided by automatic medical interview processing by the patient-side terminal, and the minimum exercise intensity may be derived based on answer information to the inquiry.

In the above-described example, the NYHA classification is exemplified as the information indicating the severity of heart failure, but the present invention is not necessarily limited thereto. For example, an American heart association/American college of cardiology (ACC/AHA) stage classification or the like may be used as the information indicating the severity. In addition, the information is not limited to such a classification, and may indicate the degree of deterioration of the body function. In the above example, the disease of medical care target is heart failure, but the medical care target is not limited thereto. For example, the present invention can also be applied to medical care for hypertensive patients.

### REFERENCE NUMERALS LIST

1, 2 . . . Medical care assistance system
100 . . . Server device
110, 210, 310 . . . Control unit
120, 240, 340 . . . Storage means
130, 250, 350 . . . Communication means
200 . . . Physician-side terminal
220, 320 . . . Input means
230, 330 . . . Output means
300 . . . Patient-side terminal
400 . . . Measuring device
P . . . Patient
N . . . Communication network
OV . . . Summary information region
MM . . . Minimum METs information
W . . . Weight information region
NT . . . Estimated NYHA classification transition region
SB . . . Estimated severity display bar
S . . . Subjective symptom information region
ME . . . Medication information region
BP . . . Blood pressure information region
HP . . . Heart rate/pulse rate information region

1. A medical care assistance system comprising:
a minimum exercise intensity calculation device configured to obtain a minimum exercise intensity that is an exercise intensity of an exercise having the smallest exercise intensity among exercises that make a symptom related to a disease of medical care target for a patient to appear within a first predetermined period;
an estimated severity information calculation device configured to obtain, based on the minimum exercise intensity, estimated severity information indicating estimated severity of a disease of the patient;
a medical care assistance image generation device configured to generate a medical care assistance image including an estimated severity time series graph indicating, as a time axis, a second predetermined period that is a past predetermined period including the first predetermined period, and indicating, along the time axis, an estimated severity display bar such that the estimated severity information of a period is identifiably characteristically displayed for each of the first predetermined period or an alternative first predetermined period that is a period alternative to the first predetermined period; and
an output device configured to output the medical care assistance image.

2. The medical care assistance system according to claim 1, further comprising a symptom appearing exercise information acquisition device configured to acquire symptom appearing exercise information which is information including a content of exercise that caused a symptom related to the disease to appear, wherein
the minimum exercise intensity calculation device calculates the minimum exercise intensity based on the symptom appearing exercise information.

3. The medical care assistance system according to claim 2, wherein
the symptom appearing exercise information acquisition device further acquires information regarding the presence or absence of the symptom related to the disease of the patient for each third predetermined period, and
the medical care assistance image generation device generates the medical care assistance image displaying, along the time axis, information regarding the presence or absence of the symptom for each of the third predetermined periods.

4. The medical care assistance system according to claim 3, wherein
in the estimated severity time series graph, a length of a period indicated by one of the estimated severity display bars has the third predetermined period as a minimum value.

5. The medical care assistance system according to claim 2, further comprising an automatic medical interview terminal configured to execute an automatic medical interview processing of requesting the patient to input patient information including at least the symptom appearing exercise information of the patient within the most recent first predetermined period, wherein
the symptom appearing exercise information acquisition device acquires the symptom appearing exercise information input from the patient through an automatic medical interview processing executed in the automatic medical interview terminal.

6. The medical care assistance system according to claim 5, wherein
when an interval of inputs between two or more inputs of the symptom appearing exercise information performed by the patient exceeds the first predetermined period,
the medical care assistance image generation device displays the estimated severity display bar with a length of the first predetermined period backward from a timing at which a subsequent input of the symptom appearing exercise information is performed.

7. The medical care assistance system according to claim 5, wherein
when an interval of inputs between two or more inputs of the symptom appearing exercise information performed by the patient is shorter than the first predetermined period,
the medical care assistance image generation device sets the interval as the alternative first predetermined period, and displays the estimated severity display bar with a length of the alternative first predetermined period backward from a timing at which a subsequent input of the symptom appearing exercise information is performed.

8. The medical care assistance system according to claim 1, wherein
the medical care assistance image generation device generates the medical care assistance image in that the estimated severity display bar is displayed in color-coded manner according to the estimated severity information and text information related to the minimum exercise intensity and/or the estimated severity information of the patient in the first predetermined period is indicated by superimposing on the estimated severity display bar or in the vicinity of the estimated severity display bar.

9. The medical care assistance system according to claim 1, wherein
the disease of medical care target is heart failure, and the estimated severity information is assumed to be NYHA classification.

10. A medical care assistance device comprising: the minimum exercise intensity calculation device; the estimated severity information calculation device; and the medical care assistance image generation device, the medical care assistance device constituting at least a part of the medical care assistance system according to claim 1.

11. A recording medium for causing a computer to function as the medical care assistance device according to claim 10.
